(19) United States
(12) Patent Application Publication
(10) Pub. No.: US 2023/0183366 A1
(43) Pub. Date: Jun. 15, 2023

(54) RECOMBINANT PROTEINS WITH OX40 ACTIVATING PROPERTIES

(71) Applicants: INSERM (INSTITUT NATIONAL DE LA SANTÉ ET DE LA RECHERCH MÉDICALE), Paris (FR); UNIVERSITE PARIS EST CRETEIL VAL DE MARNE, Creteil (FR); BAYLOR RESEARCH INSTITUTE, Dallas, TX (US); ASSISTANCE PUBLIQUE - HOPITAUX DE PARIS, Paris (FR)

(72) Inventors: Yves LEVY, Creteil (FR); Sandra ZURAWSKI, Dallas, TX (US); Gérard ZURAWSKI, Dallas, TX (US)

(21) Appl. No.: 17/998,611

(22) PCT Filed: May 11, 2021
(86) PCT No.: PCT/EP2021/062453

§ 371 Date: Nov. 11, 2022

(30) Foreign Application Priority Data
May 13, 2020 (EP) 20305487.9

(51) Int. Cl. C07K 16/28; A61P 35/00

(52) U.S. Cl. CPC C07K 16/2878; A61P 35/00; C07K 2317/565

(57) ABSTRACT

The disclosure relates to the field of OX40 activating proteins. More specifically, it is disclosed herein recombinant proteins with OX40 agonist antibodies or their antigen-binding fragments fused or linked to OX40 ligand. Also disclosed is the advantageous use of such OX40 activating proteins, in particular for inducing immune responses directed to delivered antigens such as viral or cancer antigens, and/or for treating cancer.

The disclosure relates to the field of OX40 activating proteins. More specifically, disclosed herein are recombinant proteins based on OX40 agonist antibodies with their antigen-binding fragments fused or linked to OX40 ligand (OX40L). Also disclosed is the advantageous use of such OX40 activating proteins, in particular for treating cancer by boosting immune response against cancer cells.

### BACKGROUND

OX40 (also known as TNFRSF4 or CD134) is transiently expressed by CD4⁺ and CD8⁺ T cells following TCR stimulation, and interacts with OX40L expressed on activated dendritic cells to enhance proliferation and expression of effector molecules and cytokines by these T cells (reviewed in Buchan et al., 2018). Thus both OX40L-Fc fusion protein and agonistic anti-OX40 antibodies are under clinical study for boosting immunity against e.g., cancer (Linch et al., 2015, Aspeslaghab et al., 2016).

Agonistic anti-OX40 antibodies are typically configured as IgG1 to facilitate crosslinking of to FcγR receptors to help cluster surface OX40 enabling activation of downstream signaling pathways (Voo et al., 2013; Zhang et al., 2017; Gonzalez at al. 2017). However, soluble versions of OX40L trimer can act independently of Fc cross-linking (Ito et al., 2006).

The inventors selected a clinical candidate anti-OX40 mAb agonist and fused OX40L to this mAb. Surprisingly, they have noticed a remarkable improvement for the in vitro potency of such fusion protein compared to the OX40L IgG1 by >2 logs; the anti-OX40 mAb activity also became independent of Fc cross-linking; and the efficacy (maximum response) increased.

Such OX40 activating proteins may thus be of great value in therapy, in particular immunotherapy for treating cancer, and to adjuvant immune responses directed to administered antigens.

### SUMMARY

The present disclosure provides an OX40 activating protein comprising at least the following protein domains:
an OX40 agonist antibody or an antigen-binding fragment thereof (αOX40); and,
the OX40 binding domain of OX40L (OX40L).

In certain embodiments, said OX40 agonist antibody or its antigen-binding fragment binds specifically to human OX40 and has at least one or more of the following properties:
(i) it induces the proliferation of T cells, as measured in vitro by flow cytometric analysis, e.g. by analysis of replicative dilution of CFSE-labelled cells; or,
(ii) it induces the secretion of cytokines from T cells, such as IL5, IL13, IFNγ and/or TNFα cytokine as measured in vitro with a CD4+ T cell activation assay.

In specific embodiments, said binding domain of OX40L is a fragment of OX40L comprising SEQ ID NO:2.

In certain embodiments, said binding-domain of OX40L is fused to the C-terminus of a light or heavy chain of said OX40 agonist antibody or its antigen-binding fragment.

In a preferred embodiment, the OX40 activating protein as disclosed herein comprises heavy and light chains of OX40 agonist IgG antibody.

In certain embodiments, said OX40 activating protein may further comprise a peptide linker between OX40L and the light or heavy chain of said OX40 agonist antibody or its antigen-binding fragment, preferably a flexible linker FlexV1 of SEQ ID NO:13.

In specific embodiments, said OX40 agonist antibody is selected from the following antibodies:
(i) an antibody comprising the HCDR1 of SEQ ID NO:3, HCDR2 of SEQ ID NO:4, HCDR3 of SEQ ID NO:5, LCDR1 of SEQ ID NO:6, LCDR2 of SEQ ID NO:7 and LCDR3 of SEQ ID NO:8;
(ii) an antibody comprising VH and VL domains of SEQ ID NO 9 and SEQ ID NO:10 respectively;
(iii) an antibody that competes for binding to OX40 expressing cells with at least one of the antibodies identified in (i) or (ii); or,
(iv) an antibody that binds to the same epitope as one of the antibodies identified in (i) or (ii).

In certain embodiments, one or more antigens are fused to the heavy or light chain of said OX40 agonist antibody or its antigen-binding fragment. Typically, said one or more viral or cancer antigens are fused to the heavy or light chain of an OX40 agonist antibody.

The OX40 activating protein may comprise for example, a light chain of the formula αOX40Light-PL-OX40L, and a heavy chain of the formula αOX40Heavy-(PL-Ag)x, wherein
αOX40Light is a light chain of said OX40 agonist antibody;
αOX40Heavy is a heavy chain of said OX40 agonist antibody;
PL is a bond or a peptide linker, either identical or different, typically FlexV1 of SEQ ID NO:15;
Ag is a viral or cancer antigen, either identical or different;
x is 0 or is an integer from 1 to 20, for example from 1, 2, 3, 4, or 5;
OX40L is the binding domain of the ligand of OX40 comprising SEQ ID NO:2; and,
is absent when x is 0, or is a bond.

The disclosure further relates to a pharmaceutical composition, comprising said OX40 activating protein as herein described, and one or more pharmaceutically acceptable excipients.

The OX40 activating protein as described herein may advantageously be used in treating cancer in a subject in need thereof.

In other embodiments, the OX40 activating protein as described herein may be useful in eliciting T cell proliferation and/or inducing cytokine proliferation of T cells in a subject.

### DETAILED DESCRIPTION

### Definitions

In order that the present disclosure may be more readily understood, certain terms are first defined. Additional definitions are set forth throughout the detailed description.

As used herein, the term “OX40” has its general meaning in the art and refers to human OX40 polypeptide receptor including OX40 of SEQ ID NO:1. In specific embodiments, OX40 is the human canonical sequence as reported by UniProtKB-P43489 (also referred as human TNFRSF4 or CD134). The ectodomain of OX40 which is recognized by certain anti-OX40 antibodies may typically be SEQ ID NO:18.

As used herein, the term “OX40L” (also known as CD134L or TNFSF4) has its general meaning in the art and refers to human OX40L polypeptide, for example, as reported by UniProtKB-P23510, including its OX40-binding domain of SEQ ID NO:2. OX40L may be expressed as a soluble polypeptide and is the natural ligand of OX40 receptor.

As used herein, the term “protein” refers to any organic compounds made of amino acids arranged in one or more linear chains (also referred as “polypeptide chains”) and folded into a globular form. The amino acids in such polypeptide chain are joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, amino acid modification for chemical conjugation or other molecule, etc. . . .

As used herein, a “complex protein” refers more specifically to a protein which is made of at least two polypeptide chains, wherein said at least two polypeptide chains are associated together under appropriate conditions via either non-covalent binding or covalent binding, for example, by disulphide bridge.

A “heterodimeric protein” refers to a protein that is made of at least two polypeptide chains, forming a complex protein, wherein said two polypeptide chains have different amino acid sequences.

The terms “polypeptide,” “peptide” and “protein” expressly include glycoproteins, as well as non-glycoproteins. In specific embodiments, the term “polypeptide” and “protein” refers to any polypeptide or protein that can be encoded by a gene and translated using cell expression system and DNA recombinant means, such as mammalian host cell expression system.

The term “recombinant protein”, as used herein, includes proteins that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) fusion proteins isolated from a host cell transformed to express the corresponding protein, e.g., from a transfectoma, etc. . . .

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins.

The term “antibody” as used herein refers to immunoglobulin molecules and immunologically active portions of immunoglobulin molecules, i.e., molecules that contain an antigen binding site that immunospecifically binds to an antigen.

In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence» within the Antibodies pages. The predicted CDRs of some agonist antibodies, such as 11B6, 12E2, 12B4, CP (CP-870,893 from Pfizer) or 24A3 are described in the Examples below.

The term “antigen-binding fragment” of an antibody (or simply “antibody fragment”), as used herein, refers to full length or one or more fragments of an antibody that retain the ability to specifically bind to an antigen (e.g., the ectodomain of OX40). It has been shown that the antigen-binding function of an antibody can be performed by fragments of a full-length antibody. Examples of binding fragments encompassed within the term “antigen-binding fragment” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain, or any fusion proteins comprising such antigen-binding fragments.

Furthermore, although the two domains of the Fv fragment, V_{L} and V_{H}, are coded for by separate genes, they can be joined, using recombinant methods, by a synthetic linker that enables them to be made as a single chain protein in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody. These antibody fragments are obtained using conventional techniques known to those of skill in the art, and the fragments are screened for utility in the same manner as are intact antibodies.

As used herein, the term “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction.

The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art.

A method for determining the K_{D} of a protein or an antibody is by using surface plasmon resonance, for example by using a biosensor system such as a Biacore® system.

As used herein, the term “binding specificity” refers to the ability of an antibody to detectably bind to an antigen recombinant polypeptide, such as recombinant OX40 polypeptide, with a K_{D} of 100 nM or less, 10 nM or less, 5 nM or less, as measured by Surface Plasmon Resonance (SPR) measurements, for example as determined in the Examples.

An antibody that “does not cross-react with a particular antigen” is intended to refer to an antibody that binds to that antigen, with a K_{D} of 100 nM or greater, or a K_{D} of 1 mM or greater, or a K_{D} of 10 mM or greater, said affinity being measured for example using similar Surface Plasmon Resonance (SPR) measurements, as disclosed in the Examples. In certain embodiments, such antibodies that do not cross-react with the antigen exhibit essentially undetectable binding against these proteins in standard binding assays.

The isolated OX40 activating protein according to the present disclosure is a protein that has binding specificity to OX40 and activating or agonist properties with respect to OX40 receptor. An OX40 activating protein may have cross-reactivity to other antigens, such as related OX40 molecules from other species. Moreover, in specific embodiments, an isolated OX40 activating protein may be substantially free of other cellular material and/or chemicals.

The phrases “an antibody recognizing an antigen” and “an antibody having specificity for an antigen” are used interchangeably herein with the term “an antibody which binds specifically to an antigen”.

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a OX40 polypeptide). The term “affinity”, as used herein, means the strength of the binding of an antibody to an epitope.

The present disclosure relates to the unexpected finding that fusion proteins of OX40L with certain OX40 agonist antibodies (e.g., derived from agonist mAb 24 as described in U.S. Pat. No. 9,738,723B2), exhibit superior OX40 activating properties compared to the corresponding agonist antibody alone or the combined administration of such agonist antibody with soluble OX40L (sOX40L).

“Humanized antibody” as used herein, refers broadly to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies may include amino acid residues not encoded by human germline immunoglobulin sequences (e.g., mutations introduced by random or site-specific mutagenesis in vitro or by somatic mutation in vivo), for example in the CDRs. The antibodies as used herein may include amino acid residues not encoded by human sequences (e.g., mutations introduced by random or site-specific mutagenesis in vitro or by somatic mutation in vivo). In particular, the term “humanized antibody” include antibodies that comprise a silent variant of Fc IgG region.

In specific embodiments, the term «humanized antibody» include antibodies which have the 6 CDRs of a murine antibody, but humanized framework and constant regions.

More specifically, the term “humanized antibody”, as used herein, may include antibodies in which CDR sequences derived from the germline of another mammalian species, such as a mouse, have been grafted onto human framework sequences.

As used herein, a «OX40 agonist» antibody is intended to refer to an antibody that enhance OX40 mediated signaling activity in the absence of OX40L in a cell-based assay, such as the activated CD4+ T cell proliferation or cytokine production assay. Such assays are described in more details in the examples below.

As used herein, the term “silent” antibody refers to an antibody that exhibits no or low ADCC activity. As used herein, the term “ADCC” or “antibody dependent cell cytotoxicity” activity refers to cell depleting activity. ADCC activity can be measured by ADCC assays as described in the literature.

Silenced effector functions can be obtained by mutation in the Fc region of the antibodies and have been described in the Art: Strohl 2009 (LALA & N297A); Baudino 2008, D265A (Baudino et al., J. Immunol. 181 (2008): 6664-69, Strohl, CO Biotechnology 20 (2009): 685-91). Examples of silent Fc IgG1 antibodies comprise L234A and L235A mutations in the IgG1 Fc amino acid sequence.

As used herein, a protein or antibody with «OX40 activating» properties refers to a protein or antibody that is able to increase OX40 mediated signaling activity. In particular, as used herein, a protein with OX40 activating properties has at least one or more of the following properties:
(i) it induces the proliferation of T cells, as measured in vitro by flow cytometric analysis, e.g. by analysis of replicative dilution of CFSE-labelled cells, more preferably as described in the Examples below; or,
(ii) it induces the secretion of cytokines from T cells, such as IL5, IL13, IFNγ and/or TNFα cytokine as measured in vitro with a CD4+ T cell activation assay, typically as determined in the Examples below.

In specific embodiment, said OX40 activating protein of the present disclosure has the above property (i) and/or (ii) that are equal or higher than a soluble version of OX40L, the natural ligand of OX40 receptor.

In specific embodiments, said OX40 activating protein includes a OX40 binding domain of OX40L which is not a trimeric form.

In specific embodiments, said OX40 activating protein of the present disclosure is tetravalent with respect to OX40 binding.

In specific embodiments, said OX40 activating protein includes a bivalent OX40 agonist antibody with one monomeric OX40 binding domain of OX40L covalently or non-covalently bound to each arm of said bivalent antibody, preferably via the C-terminal part of each arm of the bivalent antibody, either the light chain or heavy chain of each arm.

In other specific embodiments, said OX40 activating protein of the present disclosure has activating properties at least 2 fold, 3 fold, 5 fold, 10 fold, or at least 50 fold more active than a reference OX40 agonist antibody being typically selected among the following OX40 agonist antibodies: mAb 24 as described in U.S. Pat. No. 9,738,723B2, typically as determined with 1 nM antibody in the CD4+ T cell activation assay as described in the Examples.

In other specific embodiment, said OX40 activating protein of the present disclosure has equal or higher activating properties than a combined composition of soluble sOX40L co-administered with the same OX40 agonist antibody (or its antigen-binding fragment) as present in said OX40 activating protein.

As used herein, the term, “optimized” means that a nucleotide sequence has been altered to encode an amino acid sequence using codons that are preferred in the production cell or organism, generally a eukaryotic cell, for example, a Chinese Hamster Ovary cell (CHO) or a human cell. The optimized nucleotide sequence is engineered to retain completely or as much as possible the amino acid sequence originally encoded by the starting nucleotide sequence. The amino acid sequences encoded by optimized nucleotide sequences are also referred to as optimized.

As used herein, the percent identity between the two sequences is a function of the number of identical positions shared by the sequences (i. e., % identity=number of identical positions/total number of positions x 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, “Dendritic Cells” (DCs) refers to any member of a diverse population of morphologically similar cell types found in lymphoid or non-lymphoid tissues. These cells are characterized by their distinctive morphology and high levels of surface MHC-class II expression. These cells can be isolated from a number of tissue sources, and conveniently, from peripheral blood, as described in the Examples below.

The OX40 Activating Protein of the Present Disclosure

The present disclosure relates to an OX40 activating protein comprising at least the following protein domains:
(i) an OX40 agonist antibody (αOX40) or an antigen-binding fragment thereof; and,
(ii) the OX40 binding domain of OX40L (OX40L), typically of SEQ ID NO:2 or a functional fragment thereof with at least 90%, 95% or 100% identity to SEQ ID NO:2.

In certain embodiments, the OX40 binding domain of OX40L (preferably as a monomeric form) is covalently or non-covalently attached to the C-terminus of a light or heavy chain of said OX40 agonist antibody or its antigen-binding fragment, optionally via a linker, such as a peptidic or chemical linker. In one embodiment, the OX40 binding-domain of OX40L is non-covalently attached to the C-terminus of the light chain of a OX40 agonist antibody or its antigen-binding fragment.

In certain embodiments, the OX40 binding domain of OX40L is fused to the C-terminus of a light or heavy chain of said OX40 agonist antibody or its antigen-binding fragment, optionally via a linker, such as a peptidic linker. Typically, the OX40 binding domain of OX40L is fused to the C-terminus of the light chain of a OX40 agonist antibody or its antigen-binding fragment, optionally via a linker, such as a peptidic linker.

In other specific embodiments, said OX40 binding domain of OX40L is conjugated to the OX40 agonist antibody or its antigen-binding fragment using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

In certain embodiments, said OX40 activating protein as disclosed herein is an antibody-like protein comprising a light chain of the formula αOX40Light-PL-OX40L and a heavy chain of the formula αOX40Heavy, wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
OX40L is the OX40 binding domain of OX40 ligand, for example comprising SEQ ID NO:2, or a functional fragment thereof with at least 90%, 95% or 100% identity to SEQ ID NO:2.

In a more specific embodiment, said OX40 activating protein as disclosed herein is an antibody-like protein comprising a light chain of the formula αOX40Light-PL-OX40L and a heavy chain of the formula αOX40Heavy-(PL-Doc), wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
Doc is a dockerin domain or multiple domains to permit non-covalent coupling to cohesin fusion proteins as described in US20160031988A1 and US20120039916A1, for example comprising SEQ ID NO:111.

In other specific embodiments, said OX40 activating protein as disclosed herein is an antibody-like protein comprising a heavy chain of the formula αOX40Heavy-PL-OX40L and a heavy chain of the formula αOX40Light, wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
OX40L is the OX40-binding domain of OX40 ligand, for example comprising SEQ ID NO:2, or its functional fragment with at least 90%, 95% or 100% identity to SEQ ID NO:2.

In a other more specific embodiment, said OX40 activating protein as disclosed herein is an antibody-like protein comprising a heavy chain of the formula αOX40Heavy-PL-CD40L and a heavy chain of the formula αOX40Light-(PL-Doc)x, wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
Doc is a dockerin domain or multiple domains to permit non-covalent coupling to cohesin fusion proteins as described in US20160031988A1 and US20120039916A1.

Preferred embodiments of αOX40Light, αOX40Heavy, and OX40L are further described in the next sections.

In specific embodiments, the OX40 activating protein of the present disclosure refers to a complex protein comprising two heterodimers, each heterodimer consisting of one heavy and one light chains of amino acids, stably associated together, for example via one or more disulfide bonds. Typically, the heavy chain comprises at least the V_{H} region, preferably at least the CH1-V_{H} regions of a OX40 agonist antibody and the light chain comprises at least the VL region, preferably at least the CL-VL regions of said OX40 agonist antibody. At least, said heavy or light chain is fused or conjugated to at least the OX40 binding domain of OX40L, optionally via a linker, for example a peptidic linker.

In specific embodiments, said OX40 activating protein of the present disclosure comprises heavy and light chains of a OX40 agonist IgG antibody, including isotype constant region or IgG Fc region, for example IgG1 or IgG4, or a mutated silent IgG Fc.

In a more specific embodiment, said OX40 activating protein as disclosed herein is an antibody-like protein comprising a light chain of the formula αOX40Light-PL-OX40L and a heavy chain of the formula αOX40Heavy-(PL-Ag)x, wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
Ag is a viral or cancer antigen, either identical or different;
x is 0 or is an integer from 1 to 20, for example from 1, 2, 3, 4, or 5;
OX40L is the binding domain of the ligand of OX40 comprising SEQ ID NO:2 or a functional fragment thereof with at least 90%, 95% or 100% identity to SEQ ID NO:2; and
is absent when x is 0, or is a covalent bond.

In another more specific embodiment, said OX40 activating protein as disclosed herein is an antibody-like protein comprising a heavy chain of the formula αOX40Heavy-PL-OX40L and a light chain of the formula αOX40Light-(PL-Ag)x, wherein
αOX40Light is a light chain of a OX40 agonist antibody;
αOX40Heavy is a heavy chain of a OX40 agonist antibody;
PL is a bond or a peptide linker, either identical or different, preferably FlexV1 of SEQ ID NO:13;
Ag is a viral or cancer antigen, either identical or different;
x is 0 or is an integer from 1 to 20, for example from 1, 2, 3, 4, or 5;
OX40L is the binding domain of the ligand of OX40 comprising SEQ ID NO:2 or a functional fragment thereof with at least 90%, 95% or 100% identity to SEQ ID NO:2; and
is absent when x is 0, or is a covalent bond.

In specific embodiments, the PL is a peptide linker preferably ensuring optimal activating properties and yield in cell production.

In specific embodiments, the -(PL-Ag)x is located at the carboxy terminus of the heavy chain of said OX40 activating antibody-like protein.

Typically, a schematic representation of an embodiment of said OX40 activating protein is shown in FIG. 1. Preferred embodiments of αOX40Light, αOX40Heavy, and OX40L are further described in the next sections.

In certain embodiments, peptide linkers may incorporate glycosylation sites or introduce secondary structure. Additionally these linkers may increase the efficiency of expression or stability of the fusion protein and as a result the efficiency of antigen presentation to a dendritic cell. Such linkers may include the flexV1, f1, f2, f3 and/or f4 linkers. These examples and others are discussed in WO 2010/104747, the contents of which are incorporated herein by reference. In particular, flexV1 is a polypeptide of SEQ ID NO:13.

In a more specific embodiment, said OX40 activating protein comprises an OX40 agonist antibody comprising V_{H} of SEQ ID NO: 9 and VL of SEQ ID NO: 10.

In another specific embodiment, said OX40 activating protein comprises an OX40 agonist antibody comprising HCDR1, HCDR2, HCDR3, LCDR1, LCDR2, and LCDR3 of SEQ ID NO:3-8 respectively.

In another specific embodiment, said OX40 activating protein consists of a heavy chain polypeptide comprising SEQ ID NO: 11 and a light chain comprising SEQ ID NO 12.

OX40 activating proteins with amino acid sequences having at least 90%, for example, at least 95%, 96%, 97%, 98%, or 99% identity to any one of the above defined amino acid sequences are also part of the present disclosure, typically OX40 activating proteins have at least equal or higher activating properties than said OX40 activating protein consisting of heavy chain of SEQ ID NO:11 and light chain of SEQ ID NO:12.

The OX40 Agonist Antibody for Use in Preparing the Fusion Protein of Present Disclosure

The skilled person may use OX40 agonist antibodies already known in the art or generate de novo novel OX40 activating antibodies using antibody screening technologies.

More specifically, said OX40 agonist antibody (or its antigen-binding fragment) for use in the OX40 activating protein of the present disclosure have one or more of the following advantageous properties:
(i) it binds to OX40 ectodomain with a K_{D} of 500 nM or less, for example between 50 or less and 500 nM, as measured by SPR binding assay, for example as described in the Examples below; typically with a K_{D} of about 131 nM;
(ii) it induces the proliferation of T cells, as measured in vitro by flow cytometric analysis, for example as measured with the CD4+ T cell proliferation assay described in the Examples below; and/or
(iii) it induces the secretion of cytokines, such as IL-13, or TNFα, as measured in vitro with a T cell activation assay as described in the Examples below.

In specific embodiment, an OX40 agonist antibody is an antibody which has OX40 mediated signaling activity in the absence of OX40L in a cell-based assay which is at least similar to the OX40 mediated signaling activity of a reference OX40 agonist antibody as measured in the same cell-based assay, for example, said reference OX40 agonist antibody may be mAb24 as described below.

To select novel OX40 agonist antibodies, a variety of methods of screening antibodies have been described in the Art. Such methods may be divided into in vivo systems, such as transgenic mice capable of producing fully human antibodies upon antigen immunization and in vitro systems, consisting of generating antibody DNA coding libraries, expressing the DNA library in an appropriate system for antibody production, selecting the clone that express antibody candidate that binds to the target with the affinity selection criteria and recovering the corresponding coding sequence of the selected clone.

These in vitro technologies are known as display technologies, and include without limitation, phage display, RNA or DNA display, ribosome display, yeast or mammalian cell display. They have been well described in the Art (for a review see for example: Nelson et al., 2010 Nature Reviews Drug discovery, “Development trends for human monoclonal antibody therapeutics” (Advance Online Publication) and Hoogenboom et al. in Method in Molecular Biology 178:1-37, O'Brien et al., ed., Human Press, Totowa, N.J., 2001). In one specific embodiment, human recombinant OX40 agonist antibodies are isolated using phage display methods for screening libraries of human recombinant antibody libraries with OX40 binding and agonist properties.

Repertoires of V_{H} and V_{L} genes or related CDR regions can be separately cloned by polymerase chain reaction (PCR) or synthesized by DNA synthesizer and recombined randomly in phage libraries, which can then be screened for antigen-binding clones. Such phage display methods for isolating human antibodies are established in the art or described in the examples below. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

In a certain embodiment, human antibodies directed against OX40 can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous p and K chain loci (see e.g., Lonberg, et al., 1994 Nature 368(6474): 856-859).

In another embodiment, human OX40 agonist antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Monoclonal antibodies (mAbs) can also be produced by conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

It is further contemplated that monoclonal antibodies may be further screened or optimized for their OX40 agonist properties as above defined. In particular, it is contemplated that monoclonal antibodies may have 1, 2, 3, 4, 5, 6, or more alterations in the amino acid sequence of 1, 2, 3, 4, 5, or 6 CDRs of monoclonal antibodies or humanized antibodies provided herein. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of antibodies may have an insertion, deletion, or substitution with a conserved or non-conserved amino acid. Such amino acids that can either be substituted or constitute the substitution are disclosed above.

OX40 agonist antibodies for use in preparing the OX40 activating proteins of the disclosure include the recombinant OX40 agonist antibodies mAb24 which are structurally characterized by their variable heavy and light chain amino acid and nucleotide sequences as described in the Tables 1 and 2 below:

TABLE 1
Variable heavy and light chain amino
acid sequences of mAb1-mAb6

| | VH | VL |
|---|---|---|
| Antibody | Amino acid sequence | Amino acid sequence |
| mAb24 | SEQ ID NO: 9 | SEQ ID NO: 10 |

TABLE 2
Variable heavy and light chain nucleotide
(nt) coding sequences of mAb1-mAb6

| | VH | VL |
|---|---|---|
| Antibody | Nt coding sequence | Nt coding sequence |
| mAb24 | SEQ ID NO: 14 | SEQ ID NO: 15 |

Other OX40 agonist antibodies which may be used include any chimeric or humanized antibodies comprising the 6 CDRs of mAb24.

Examples of the amino acid sequences of the VH CDR1s (also called HCDR1), VH CDR2s (also called HCDR2), VH CDR3s (also called HCDR1), VL CDR1s (also called LCDR1), VL CDR2s (also called LCDR2), VL CDR3s (also called HCDR3) of some OX40 agonist antibodies according to the disclosure are shown in Table 3.

In Table 3, the CDR regions of the antibodies of the present disclosure are delineated using the Kabat numbering (Kabat et al., 1992).

TABLE 3
CDR regions of mAb24 according to Kabat numbering

| Original | | | | | | |
|---|---|---|---|---|---|---|
| antibody | HCDR1 | HCDR2 | HCDR3 | LCDR1 | LCDR2 | LCDR3 |
| mAb24 | SEQ ID | SEQ ID | SEQ ID | SEQ ID | SEQ ID | SEQ ID |
| | NO: 3 | NO: 4 | NO: 5 | NO: 6 | NO: 7 | NO: 8 |

In specific embodiments, a OX40 agonist antibody is selected from the following antibodies:
(i) a humanized antibody comprising the 6 CDRs of mAb24;
(ii) a humanized antibody comprising VH and VL domains of SEQ ID NO:9 and SEQ ID NO:10 respectively;
(iii) an antibody that competes for binding to OX40 expressing cells with at least one of the antibodies identified in (i) or (ii),
(iv) an antibody that binds to the same epitope as one of the antibodies identified in (i) or (ii).

Any other known or newly developed OX40 agonist antibodies can be potentially linked with OX40L using the method revealed in the present disclosure to increase their biological activity.

Nucleic Acid Molecules Encoding the OX40 Activating Proteins of the Disclosure

Also disclosed herein are the nucleic acid molecules that encode the OX40 activating proteins of the present disclosure.

Examples of nucleic acid molecules are those encoding the variable light and heavy chain amino acid sequences of the OX40 activating antibody-like proteins as disclosed in the previous section, and using the genetic code and, optionally taking into account the codon bias depending on the host cell species.

Typically, nucleic acid molecules encoding the OX40 activating protein of the disclosure comprises coding sequences of OX40 agonist antibody consisting of SEQ ID NO 11 and SEQ ID NO 12, for example the nucleic acids of SEQ ID NO:16 and SEQ ID NO:17 respectively.

Nucleic acids encoding OX40 activating proteins of the disclosure with nucleotide sequences having at least 90%, for example, at least 95%, 96%, 97%, 98%, or 99% identity to any one of the above defined nucleotides sequences are also part of the present disclosure.

The present disclosure also pertains to nucleic acid molecules that derive from the latter sequences having been optimized for protein expression in mammalian cells, for example, CHO or HEK cell lines.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (Ausubel et al., 1988). A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding, for example, heavy and light chain segments are obtained, these DNA fragments can be further manipulated by standard recombinant DNA techniques, for example to convert the variable region genes to to Fab fragment genes or to an scFv gene.

Generation of Transfectomas Producing OX40 Activating Proteins

The OX40 activating proteins of the present disclosure can be produced in a host cell transfectoma using, for example, a combination of recombinant DNA techniques and gene transfection methods as is well known in the art (Morrison, 1985).

For example, to express the OX40 activating proteins, DNAs encoding said OX40 activating proteins (typically, full length heavy and light chains) can be obtained by standard molecular biology or biochemistry techniques (e.g., DNA chemical synthesis, PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that a gene is ligated into a vector such that transcriptional and translational control sequences within the vector serve their intended function of regulating the transcription and translation of the OX40 activating protein. The expression vector and expression control sequences are chosen to be compatible with the expression host cell used. If the OX40 activating proteins include distinct polypeptide, for example one sequence encoding a heavy chain of a OX40 activating antibody-like protein as disclosed in the above sections and another encoding a light chain of said OX40 activating antibody-like protein, the heavy and light chain encoding genes can be inserted into separate vector or, more typically, both genes are inserted into the same expression vector. The genes are inserted into the expression vector by standard methods (e.g., ligation of complementary restriction sites on the protein gene and vector, or blunt end ligation if no restriction sites are present).

Signal peptides may be further used for secretion of the polypeptides out of the expression cells, such as an immunoglobulin signal peptide or a heterologous signal peptide (i.e., a signal peptide from a non-immunoglobulin protein).

In addition, the recombinant expression vectors disclosed herein carry regulatory sequences that control the expression of the OX40 activating proteins in a host cell. The term “regulatory sequence” is intended to include promoters, enhancers and other expression control elements (e.g., polyadenylation signals) that control the transcription or translation of the respective genes. It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

Additionally, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr- host cells with methotrexate selection/amplification) and the neo gene (for G418 selection).

For expression of the OX40 activating proteins, the expression vector is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the binding proteins of the present disclosure in either prokaryotic or eukaryotic host cells. Expression of recombinant proteins in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

In one specific embodiment, a cloning or expression vector according to the disclosure comprises one or more of the nucleics acids as described in the previous section, operatively linked to suitable promoter sequences.

Mammalian host cells for expressing the recombinant antibodies of the disclosure include Chinese Hamster Ovary (CHO cells) including dhfr- CHO cells (described in Urlaub and Chasin, 1980) used with a DHFR selectable marker, CHOK1 dhfr+ cell lines, NSO myeloma cells, COS cells and SP2 cells, or HEK cells. When recombinant expression vectors encoding activating proteins genes are introduced into mammalian host cells, the activating proteins are produced by culturing the host cells for a period of time sufficient for expression of the proteins in the host cells and, optionally, secretion of the proteins into the culture medium in which the host cells are grown. The activating proteins can be recovered and purified for example from the culture medium after their secretion using standard protein purification methods.

In one specific embodiment, the host cell of the disclosure is a host cell transfected with an expression vector having the coding sequences of the OX40 activating proteins (typically coding sequence of the heavy and light chain of OX40 activating protein, for example SEQ ID NO:16 and 17) as disclosed in the previous section.

The latter host cells may then be further cultured under suitable conditions for the expression and production of said OX40 activating protein.

Pharmaceutical Compositions

In another aspect, the present disclosure provides a composition, e.g., a pharmaceutical composition, containing an OX40 activating protein, formulated together with a pharmaceutically acceptable carrier.

Pharmaceutical compositions disclosed herein also can be administered in combination therapy, i.e., combined with other agents. For example, the combination therapy can include OX40 activating protein of the present disclosure, combined with at least one anti-viral, anti-inflammatory, vaccine adjuvant and/or another anti-proliferative agent.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route. Depending on the route of administration, the active compound, i.e., OX40 activating protein, may be coated in a material to protect the compound from the action of acids and other natural conditions that may inactivate the compound.

Sterile phosphate-buffered saline is one example of a pharmaceutically acceptable carrier. Other suitable carriers are well-known to those in the art. (Remington and Gennaro, 1995). Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, parenteral, intranasal, intravenous, intramuscular, subcutaneous or intraocular administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the OX40 activating protein may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Pharmaceutically acceptable salts which may be used in the formulation include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The OX40 activating protein may be formulated within a therapeutic mixture to comprise about 0.0001 to 1.0 milligrams, or about 0.001 to 0.1 milligrams, or about 0.1 to 1.0 or even 1.0 to about 10 milligrams per dose. Multiple doses can also be administered.

Vaccine Compositions

The disclosure also relates to a vaccine comprising an OX40 activating protein of the disclosure and a pharmaceutically acceptable vehicle.

As used herein, the term “vaccine” is intended to mean a composition which can be administered to humans or to animals in order to induce an immune response; this immune response can result in a production of antibodies or simply in the activation of certain cells, in particular antigen-presenting cells, T lymphocytes and B lymphocytes. In certain embodiments the vaccine is capable of producing an immune response that leads to the production of neutralizing antibodies in the patient with respect to the antigen provided in the vaccine. The vaccine can be a composition for prophylactic purposes or for therapeutic purposes, or both.

Vaccines may include an effective amount of the OX40 activating proteins of the disclosure, dissolved or dispersed in a pharmaceutically acceptable carrier or aqueous medium. Such compositions can also be referred to as inocula. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions. The vaccine compositions of the present disclosure may include classic pharmaceutical preparations. Dispersions also can be prepared in glycerol, liquid polyethylene glycols, and mixtures thereof and in oils. Under ordinary conditions of storage and use, these preparations contain a preservative to prevent the growth of microorganisms.

In addition, if desired, the vaccine can contain minor amounts of auxiliary substances such as wetting or emulsifying agents, pH buffering agents, and/or adjuvants which enhance the effectiveness of the vaccine. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosphoryl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN-[gamma], IL-2 and IL-12) or synthetic IFN-[gamma] inducers such as poly I.C or poly ICLC (Hiltonol) can be used in combination with adjuvants described herein.

In certain embodiments, the adjuvant may be selected among poly ICLC, CpG, LPS, Immunoquid, PLA, GLA or cytokine adjuvants such as IFNα. In other embodiments the adjuvant may be a toll-like receptor agonist (TLR). Examples of TLR agonists that may be used comprise TLR1 agonist, TLR2 agonist, TLR3 agonist, TLR4 agonist, TLR5 agonist, TLR6 agonist, TLR7 agonist, TLR8 agonist or TLR9 agonist.

The vaccine preparation of OX40 activating protein as the active immunogenic ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

Methods of Use of the OX40 Activating Proteins of the Disclosure

By eliciting an immune response to the antigen(s) present in the OX40 activating proteins, the OX40 activating proteins of the disclosure may be useful as a drug, in particular for treating or preventing cancer or infectious disorders.

In some embodiments, the OX40 activating proteins may be used in a method for treating or preventing from a viral infection or cancer disorder in a subject comprising administering a therapeutically effective amount of OX40 activating protein of the disclosure to the subject.

In specific embodiments, said OX40 activating proteins may be used in a method for treating a cancer selected from a broad range of tumor types, including but not limited to the following: ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia.

The OX40 activating proteins for use in treating cancers may be used alone or in combination with additional anti-proliferative agent or immune checkpoint inhibitors, including without limitation anti-PD1, or anti-PDL1 antibodies and anti-CTLA4 antibodies (see e.g. WO2020014583).

As used herein, “treatment” is an approach for obtaining beneficial or desired clinical results. For purposes of this disclosure in relation to methods of treating cancer, beneficial or desired clinical results include, but are not limited to, one or more of the following: reducing the proliferation of (or destroying) neoplastic or cancerous cells, inhibiting metastasis of neoplastic cells, or shrinking or decreasing the size of tumor.

As used herein, an “effective dosage” or “therapeutically effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

Yet further aspects relate to a method for eliciting and/or enhancing T-cell response, for example CD4+ and/or CD8+ T cell response against a viral or tumor associated antigen, in a subject in need thereof, comprising administering to said subject in need thereof, an OX40 activating protein or vaccine of the disclosure.

Further aspects relate to a method for inducing IgG binding antibody responses to the antigens in a subject in need thereof, the method comprising administering the OX40 activating protein of the disclosure or the vaccine composition of the disclosure.

In some embodiments, the method further comprises administration of an immunostimulant. In some embodiments, the immunostimulant is administered sequentially or concomitantly to a vaccine or therapeutic composition.

In some embodiments, the immunostimulant is mixed with a vaccine composition extemporaneously prior to injection of the vaccine composition to the subject.

Additionally, the methods of the disclosure may also comprise the administration of one or more adjuvants. The adjuvants may be attached or conjugated directly or indirectly to one or more of the vaccine components, such as an antigen or OX40 activating protein. In other embodiments, the adjuvants may be provided or administered separately from the vaccine composition. In certain embodiments the adjuvant is poly ICLC, CpG, LPS, Immunoquid, PLA, GLA or cytokine adjuvants such as IFNα. In other embodiments the adjuvant may be a toll-like receptor agonist (TLR). Examples of TLR agonists that may be used comprise TLR1 agonist, TLR2 agonist, TLR3 agonist, TLR4 agonist, TLR5 agonist, TLR6 agonist, TLR7 agonist, TLR8 agonist or TLR9 agonist.

In some embodiments, the administration comprises intradermal, intramuscular, or subcutaneous administration.

In some embodiments, the viral vaccine, e.g., an OX40 activating protein comprising a viral antigen, is used in a method for potentiating an immune response to at least one viral epitope comprising administering to a patient such viral vaccine as described herein.

In some embodiments, such viral vaccine is used to prevent healthy subject to be infected by said virus, comprising administering such viral vaccine of the present disclosure, e.g. to a healthy subject, not infected by said virus (preventive treatment). In other embodiments, the viral vaccine of the present disclosure is used in a method of treating a patient in the early stages of the viral infection comprising administering to a patient said viral vaccine.

It is contemplated that at least one viral antigen elicits at least one of a humoral and/or a cellular immune response in a host, preferably a human patient or a primate.

Administration of vaccines or pharmaceutical compositions according to the present disclosure will be via any common route so long as the target tissue is available via that route in order to maximize the delivery of antigen to a site for maximum (or in some cases minimum) immune response. Administration of vaccines will generally be by orthotopic, intradermal, mucosally, subcutaneous, intramuscular, intraperitoneal or intravenous injection. Other areas for delivery include: oral, nasal, buccal, rectal, vaginal or topical. Vaccines of the disclosure are preferably administered parenterally, by injection, for example, either subcutaneously or intramuscularly.

Vaccines or pharmaceutical compositions of the present disclosure may be administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, or in the range from about 10 mg to 50 mg.

Precise amounts of active ingredient required to be administered depend on the judgment of the practitioner and may be peculiar to each subject. It will be apparent to those of skill in the art that the therapeutically effective amount of OX40 activating proteins of this disclosure will depend, inter alia, upon the administration schedule, the unit dose of antigen administered, whether the OX40 activating protein is administered in combination with other therapeutic agents, the immune status and health of the recipient, and the therapeutic activity of the particular OX40 activating protein.

A vaccine may typically be given in a single dose schedule or in a multiple dose schedule. A multiple dose schedule is one in which a primary course of vaccination may include, e.g., 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity. The course of the immunization can be followed by in vitro proliferation assays of peripheral blood lymphocytes (PBLs) co-cultured with the antigen, and by measuring the levels of IFN-[gamma] released from the primed lymphocytes. The assays may be performed using conventional labels, such as radionucleotides, enzymes, fluorescent labels and the like. These techniques are known to one skilled in the art and can be found in U.S. Pat. Nos. 3,791,932, 4,174,384 and 3,949,064.

A vaccine may be provided in one or more “unit doses”. Unit dose is defined as containing a predetermined-quantity of the vaccine calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. The amount of vaccine delivered can vary from about 0.001 to about 0.05 mg/kg body weight, for example between 0.1 to 5 mg per subject.

Further aspects relate to a kit comprising an OX40 activating protein of the disclosure, a nucleic acid of the disclosure, an expression vector of the disclosure, or a host cell of the disclosure, and; optionally, instructions for use of the kit. The kit may be used to perform the methods described herein. In some embodiments, the kit is for eliciting a CD4+ and/or CD8+ T cell response in a subject; wherein the kit comprises the OX40 activating protein of the disclosure or the vaccine of the disclosure.

The disclosure will be further illustrated by the following examples. However, these examples should not be interpreted in any way as limiting the scope of the present invention.

### DESCRIPTION OF THE FIGURES

FIG. 1: A cartoon illustrating the anti-Receptor antibody-Ligand fusion concept exemplified by anti-OX40 mAb24 OX40 IgG1. Shown are relevant domains: V_{L} are the light chain variable regions; V_{H} are the heavy chain variable regions; C_{H} are the H chain constant regions 1-3; C_{L} are the light chain constant regions; linker sequences and key disulfide bonds are represented by grey lines. The disclosure anticipate alternate form of fusion including Ligand fused to H chain C-termini and diverse antibody forms and isotypes.

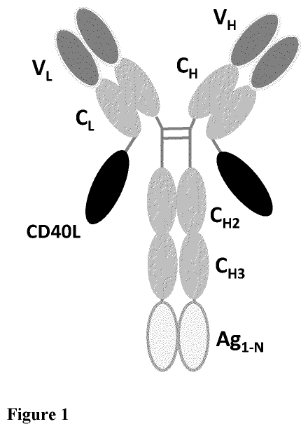

FIG. 2: Binding of anti-OX40 antibodies to human OX40-transfected CHO cells. CHO cells stably transfected with a construct for expressing human OX40 ectodomain fused to FAS transmembrane and intracellular residues were incubated at 4° C. for 30 min with a titration series of anti-OX40 IgG1, anti-OX40-OX40L IgG1, and control hIgG4. The cells were washed in PBS and probed with anti-human IgG-PE reagent at 4° C. for 30 min, washed with PBS, then 500K cells per point were analyzed by flow cytometry on a FACS Array Bioanalyzer (BD Biosciences) scoring percentage of cells brighter than the gate set for cells probed only with the detecting reagent (% P 1 of Parent).

FIG. 3. SPR analysis of soluble OX40 ectodomain binding to solid phase anti-OX40 antibodies with or without directly linked human OX40L. Cohesin-human OX40 ectodomain protein (400 nM injected for 6 min plus 5 min dissociation time at 25 μL/min) over a protein A coated surface with bound anti-OX40 IgG1 mAbs with (B) and without (A) human OX40L linked to the L chain C-termini. The cartoons show the OX40L configuration and F indicates attachment via a Flex V1 linker. Kinetic parameters are shown in Table 4. Control human IgG1 and IgG1-Flex v1-OX40L proteins run in this assay gave no or low (<6 RU) binding to OX40 in this assay.

FIG. 4. Potentiation via OX40 binding of proliferation and cytokine production by CD4+ T cells. CD4+ T cells from PBMCs of two normal donors (left panels are Donor 1; right panels are Donor 2) were primed for two days with PHA, IL-2, and anti-CD3/CD28 beads, then labeled with CFSE and cultured an additional 5 days with a titration series of the test antibodies and controls. Cells were analyzed for proliferation by flow cytometry scoring CFSE dilution (A). Culture supernatants were analyzed for IL-13 (B) and TNFα (C).

### EXAMPLES

1. Methods

Surface plasmon resonance assay. Surface plasmon resonance (SPR) assay binding measurements were performed on a SensiQ Pioneer instrument (SensiQ Technologies, Inc., Oklahoma City, Okla., USA). Protein A or Protein G (100 μg/mL in 10 mM NaAc pH4.5) were immobilized using amine coupling chemistry on COOH2 or COOH5 sensor chips at 25° C. following the manufacturer's recommended protocols. Running buffer was 10 mM HEPES, 3.4 mM EDTA, 0.005% Tween 20, 8.8 g/L NaCl, pH 7.5. Subsequently, Channel 1-2 were used to inject a dilution series of cohesin-human CD40 ectodomain (Genbank AAO43990.1 residues 22-193) or human OX40 (GenBank AAB33944.1 residues 26-211) protein (25, 12.5, 6.25, 3.125, 1.6, 0.8 nM at 25 uL/min for 2 min); finally, surfaces were regenerated through injection of 20 mM NaOH for 1 min (25 μL). The binding data were analyzed with Qdat software (SensiQ Technologies).

Assay for human OX40 binding. CHO-S cells stably transfected with a plasmid construct for expressing human OX40 ectodomain (GenBank AAB33944.1residues 26-211) fused to FAS transmembrane and intracellular domains (GenPept XP_011538069.1 residues 187-350) were incubated at 4° C. for 30 min with a titration series of anti-OX40 IgG1, anti-OX40-OX40L IgG1, and control hIgG4. The cells were washed in PBS and probed with anti-human IgG-PE reagent at 4° C. for 30 min, washed with PBS, then 500K cells per point were analyzed by flow cytometry on a FACSArray Bioanalyzer (BD Biosciences) scoring the percentage of cells brighter than the gate set for cells probed only with the detecting reagent (defined as % P1 of Parent).

Assay for OX40 agonistic activity. Blood from 2 different donors was collected in Acid-Citrate-Dextrose (ACD) tubes was processed by Ficoll-Paque® separation in SepMate™ (Stemcell Technologies, Cambridge, Mass.), using 3 ACD tubes for each SepMate™ according to the manufacturers protocol. Recovery of viable cells was in the range 1.1E8, 98.5%; and 1.13E8, 99.3%. The PBMCs were processed with the EasySep™ CD4⁺ T cell Negative Isolation Kit (Stemcell Technologies, 17952). Cells were first stimulated with PHA/IL-2/anti-CD3/CD28 beads. PHA stock (Remel purified phytohaemagglutinin, Thermo Fisher, R30852801) was in PBS, 2% FCS at 1 mg/ml and was used at 2 μg/ml. Anti-CD3/CD28 beads for polyclonal stimulation (Gibco Dynabeads Human T activator, reference 11161D, 40,000 bead per 1l) were used at 5 T cells to 1 bead. Interlekin-2 (IL-2, Pharmaceutical Grade Proleukin®) at 10E6 Units per ml was used at 20 U/ml. The isolated CD4⁺ T cells from both donors were individually stimulated at 1E6/ml in 50 ml conical tubes with 10% FCS, cRPMI and kept at 37° C. at 7% CO₂ for 48 hours. A small subset was tested by flow cytometry at 24 and 48 hours to validate increased expression of OX40 (data not shown). After 48 hours, anti-CD3/CD28 beads were removed using a magnet (Stem Cell) and cells were labeled with CFSE to enable monitoring the proliferation status using the Cell Trace Labeling Kit (Thermo Fisher, 34554) reconstituted with 18 μl DMSO to make a 5 mM stock. Cells were washed in twice in PBS warmed to 37° C. following the bead depletion. Cells were then resuspended in 1.25 μM CFSE in the warm PBS at a concentration of 1E6/ml at RT for 10 min, then quenched with 10 volumes of ice cold 10% FCS cRPMI and kept at 4° C. for an additional 5 min. Cells were washed twice in media or 2% FCS with PBS and then resuspended in media and added at 100K cells per well (50 l) into V-bottom culture plates for stimulation. Test proteins were added as a dilution series from 2 μM to 0.05 nM. Cell controls without test vehicles were included as controls and some were also stimulate with anti-CD3/CD28 (100K T cells to 40K beads, ratio of 2.5 T cells to 1 bead) as positive signal controls. Cells were then cultured an additional 5 days and culture supernatants were analyzed by Luminex® for IL-5, IL-13, IFNγ and TNFα cytokine levels (Millipore). The remaining cells were analyzed by flow cytometry (FACS CANTO, BD Biosciences) for CFSE proliferation after staining with Live/Dead™ Fixable Aqua Dead Cell Stain (Thermo Fisher) and anti-CD4 mAb using FlowJo® software (Ashland, Or).

Flow cytometry analysis. Cells were transferred in a V bottom plate, washed twice in PBS and incubated for 20 minutes at 4° C. with Live/Dead™ Fixable Aqua Dead Cell Stain Kit (Thermo Fisher Scientific, Cat. L34965) at 1:50 in a volume of 50 μL or with L/D-ef780 (Thermo Fisher, Cat. 65-0865-14) added to the antibody mix. Cells were wash twice and incubated for 30 minutes on ice with the mix of antibodies in a volume of 50 μL. Finally, cells were washed and resuspended in 200 μL BD™ stabilizing fixative (BD Biosciences, Cat. 338036) diluted 1:3. All analysis plots were pre-gated on live (using Live/Dead stain) and singlet events. Cells were analyzed with a FACS Canto II, FACSArray Bioanalyzer or a LSR Fortessa (BD Biosciences). Data was analyzed with FlowJo Software. The following antibodies were used for analysis of human DC activation: hCD80-PE, Mouse clone L307.4, ref 340294 (BD), hCD83-APC, Mouse clone HB15e, ref 551073 (BD); hCD86-FITC, Mouse clone 2331 (FUN-1), ref 555657 (BD); hCD11c-PEpCyanine7, Mouse clone 3.9, ref 25011642, (eBioscience); hHLA-DR-V450, Mouse clone G46-6, ref561359 (BD); hCD40-PE-Cyanine5, Mouse clone 5C3, ref 555590 (BD). hCD19-APC, mouse clone HIB19, ref 555415 (BD) and hCD3-PerCP, Mouse clone SK7, ref 347344 (BD) were used in the human B cell proliferation panel. The following antibodies were used for the mouse ex vivo cell analyses: mB220-AF488, clone RA3-6B2, ref 103225 (BD); mOX40L-PerCPCy5.5, clone RM134L, ref 65-5905-U025 (Tonbo Biosciences); mCD69-PE, clone H1.2F3, ref 104508 (Biolegend); mCD11c-PECy7, clone N418, ref 117318, (Biolegend); mLangerin-APC, clone 4C7, ref144206 (Biolegend); mMHCII-AF700, clone M5/114.15.2, ref 107622 (Biolegend); mCD11b-PacificBlue, clone M1/70, ref 101224 (Biolegend); mLy6G-BV510, clone 1A8, ref 127633 (Biolegend); mCD86-BV605, clone GL1, ref 105037 (Biolegend); mIgD-BV711, clone 11-26c.2a, ref 405731 (Biolegend); FC bloc, clone 2.4G2, ref 70-0161-U500 (Tonbo Biosciences).

Statistical analyses. Data are presented as means (±SEM). Statistical significance was determined by Student's t test with or without Welch's correction. A P value <0.05 was considered as statistically significant. GraphPad Prism software was used for statistical calculations.

2. Results

Fusing OX40 Ligand to an Agonistic OX40 Antibody Improves Agonist Potency and Efficacy

We linked human OX40L to the light chain C-terminus of an agonistic anti-OX40 IgG1 antibody in clinical development (humanized OX40mAb24, U.S. Pat. No. 9,738,723).

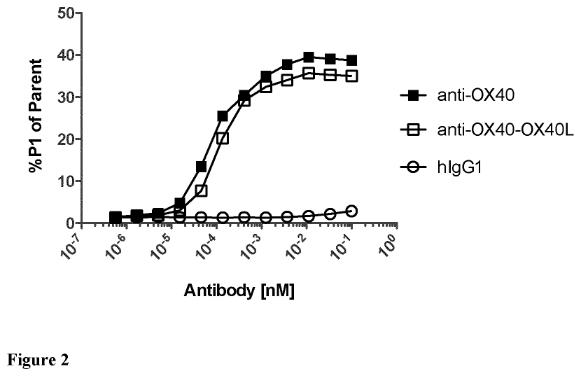

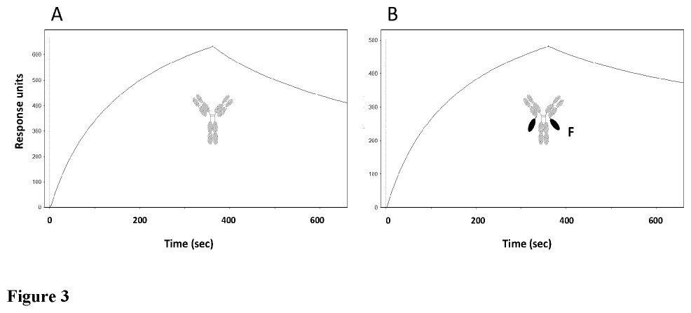

FIG. 2 shows that both the humanized anti OX40 Ab24 IgG1 and anti OX40 Ab24-OX40L fusion IgG1 proteins bound similarly to CHO cells expressing the human OX40 ectodomain. However, SPR analysis detected an ˜2-fold enhancement in off-rate associated with the addition of OX40L to the anti-OX40 mAb L chain (FIG. 3 and Table 4).

TABLE 4
Kinetic parameters and affinity constants for the interaction
between immobilized anti-human OX40 IgG1 mAbs and liquid
phase soluble human OX40 ectodomain as shown in FIG. 3.

| mAh | Ka (M⁻¹s⁻¹) | kd (s⁻¹) | KD (nM) | |
|---|---|---|---|---|
| Anti-OX40 IgG1 | 1.4E+04 [1.0] | 1.9E−03 [1.0] | 131 | [1] |
| Anti-OX40-FlexV1- | 1.6E+04 [1.1] | 8.7E−04 [2.2] | 55 | [0.42] |
| OX40L IgG1 | | | | |

Anti-OX40 hIgG1, anti-OX40-OX40L hIgG1 and hIgG1-OX40L proteins were tested for their ability to evoke proliferation and cytokine production of primed human CD4⁺ T cells in vitro. In the absence of any Fc cross-linking agent, picomolar levels of anti-OX40-OX40L were sufficient to drive both proliferation and cytokine production in this assay. In contrast, nanomolar amounts of hIgG1-OX40L were required to drive proliferation and cytokine production, while anti-OX40 was inactive in this assay.

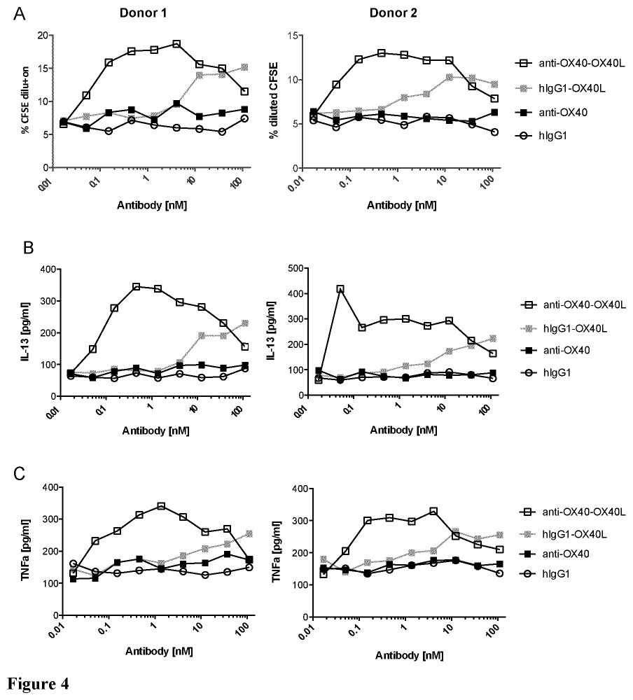

These data (FIG. 4) show that fusing OX40L to agonistic OX40 antibody profoundly changes the characteristics of the separate entities: i) the anti-OX40-OX40L IgG1 fusion increases potency of the OX40L IgG1 by >2 logs; ii) the efficacy (maximum response) is increased; and iii) the activity is independent of Fc cross-linking.

In conclusion, we selected a clinical candidate anti-X40 mAb agonist and fused OX4L to this mAb. This improved in vitro potency compared to the OX40L IgG1 by ≥2 logs; the anti-X40 mAb activity became independent of Fc cross-linking; and the efficacy (maximum response) increased.

Tables 5 and 6: Useful Sequences for Practicing the Invention

| SEQ ID | Type | Brief description |
|---|---|---|
| 1 | aa | Full amino acid sequence of OX40 |
| 2 | aa | Amino acid sequence of OX40 binding domain of human |
| | | OX40L |
| 3 | aa | HCDR1 amino acid sequence of mAb24 OX40 agonist |
| | | antibody |
| 4 | aa | HCDR2 amino acid sequence of mAb24 OX40 agonist |
| | | antibody |
| 5 | aa | HCDR3 amino acid sequence of mAb24 OX40 agonist |
| | | antibody |
| 6 | aa | LCDR1 amino acid sequence of mAb24 OX40 agonist |
| | | antibody |
| 7 | aa | LCDR2 amino acid sequence of mAb24 OX40 agonist |
| | | antibody |
| 8 | aa | LCDR3 amino acid sequence of mAb24 OX40 agonist |
| | | antibody |
| 9 | aa | VH amino acid sequence of mAb24 OX40 agonist |
| | | antibody |
| 10 | aa | VL amino acid sequence of mAb24 OX40 agonist |
| | | antibody |
| 11 | aa | Amino acid sequence of anti-OX40-OX40L IgG1 heavy |
| | | chain |
| 12 | aa | Amino acid sequence of anti-OX40-OX40L light chain |
| 13 | aa | Amino acid sequence of FlexV1 peptidic linker |
| 14 | nt | VH nucleotide sequence of mAb24 OX40 agonist |
| | | antibody |
| 15 | nt | VL nucleotide sequence of mAb24 OX40 agonist |
| | | antibody |
| 16 | nt | Nucleotide sequence of anti-OX40-OX40L IgG1 heavy |
| | | chain |
| 17 | nt | Nucleotide sequence of anti-OX40-OX40L light chain |
| 18 | aa | Ectodomain of OX40 |

| SEQ | | |
|---|---|---|
| ID | Type | SEQUENCE |
| 1 | aa | RCCHECRPGNGMVSRCSRSQNTVCRPCGPGFYNDVVSSKP |
| | | CKPCTWCNLRSGSERKQLCTATQDTVCRCRAGTQPLDSYK |
| | | PGVDCAPCPPGHFSPGDNQACKPWTNCTLAGKHTLQPASN |
| | | SSDAICEDRDPPATQPQETQGPPARPITVQPTEAWPRTSQ |
| | | GPSTRPVEVPGGRAVAAILGLGLVLGLLGPLAILLALYLL |
| | | RRDQRLPPDAHKPPGGGSFRTPIQEEQADAHSTLAKI |
| 2 | aa | ASHFSALQVSHRYPRIQSIKVQFTEYKKEKGFILTSQKED |
| | | EIMKVQNNSVIINCDGFYLISLKGYFSQEVNISLHYQKDE |
| | | EPLFQLKKVRSVNSLMVASLTYKDKVYLNVTTDNTSLDDF |
| | | HVNGGELILIHQNPGEFCVLAS |
| 3 | aa | GGSFSSGYWN |
| 4 | aa | YISYNGITYHNPSLKS |
| 5 | aa | YKYDYDGGHAMDY |
| 6 | aa | RASQDISNYLN |
| 7 | aa | YTSKLHS |
| 8 | aa | QQGSALPWT |
| 9 | aa | QVQLQESGPGLVKPSQTLSLTCAVYGGSFSSGYWNWIRKH |
| | | PGKGLEYIGYISYNGITYHNPSLKSRITINRDTSKNQYSL |
| | | QLNSVTPEDTAVYYCARYKYDYDGGHAMDYWGQGTLVTVS |
| | | SASTKGPSVFPLAPCSRSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNHY |
| | | TQKSLSLSPGKAS |
| 10 | aa | DIQMTQSPSSLSASVGDRVTITCRASQDISNYLNWYQQKP |
| | | GKAPKLLIYYTSKLHSGVPSRFSGSGSGTDYTLTISSLQP |
| | | EDFATYYCQQGSALPWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECAS |
| 11 | aa | QVQLQESGPGLVKPSQTLSLTCAVYGGSFSSGYWNWIRKH |
| | | PGKGLEYIGYISYNGITYHNPSLKSRITINRDTSKNQYSL |
| | | QLNSVTPEDTAVYYCARYKYDYDGGHAMDYWGQGTLVTVS |
| | | SASTKGPSVFPLAPCSRSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNHY |
| | | TQKSLSLSPGKAS |
| 12 | aa | DIQMTQSPSSLSASVGDRVTITCRASQDISNYLNWYQQKP |
| | | GKAPKLLIYYTSKLHSGVPSRFSGSGSGTDYTLTISSLQP |
| | | EDFATYYCQQGSALPWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECASQTPTNTISVTPTNNSTPTNNSNPK |
| | | PNPASHFSALQVSHRYPRIQSIKVQFTEYKKEKGFILTSQ |
| | | KEDEIMKVQNNSVIINCDGFYLISLKGYFSQEVNISLHYQ |
| | | KDEEPLFQLKKVRSVNSLMVASLTYKDKVYLNVTTDNTSL |
| | | DDFHVNGGELILIHQNPGEFCVLAS |
| 13 | aa | ASQTPTNTISVTPTNNSTPTNNSNPKPNPAS |
| 14 | nt | ATGGATCCTAAGGGCTCTCTGTCTTGGAGAATCCTGCTGT |
| | | TTCTGTCTCTGGCCTTTGAGCTGTCTTACGGCCAGGTGCA |
| | | GCTGCAGGAGTCTGGACCTGGCCTGGTGAAGCCTTCTCAG |
| | | ACCCTGTCTCTGACATGTGCCGTGTACGGAGGCTCTTTTT |
| | | CTTCTGGCTACTGGAACTGGATTAGAAAGCACCCTGGAAA |
| | | GGGCCTGGAGTACATTGGCTACATCTCTTACAACGGCATC |
| | | ACATATCATAACCCTTCTCTGAAGTCTAGAATCACAATTA |
| | | ACAGAGATACATCTAAGAACCAGTACTCTCTGCAGCTGAA |
| | | CTCTGTGACACCTGAGGATACAGCCGTGTACTACTGTGCC |
| | | AGATACAAGTACGATTATGATGGCGGCCACGCTATGGATT |
| | | ACTGGGGACAGGGAACACTGGTGACCGTGTCTTCTGCTTC |
| | | TACAAAGGGCCCATCGGTCTTCCCCCTGGCGCCCTGCTCC |
| | | AGGAGCACCTCTGGGGGCACAGCGGCCCTGGGCTGCCTGG |
| | | TCAAGGACTACTTCCCCGAACCGGTGACGGTGTCGTGGAA |
| | | CTCAGGCGCCCTGACCAGCGGCGTGCACACCTTCCCGGCT |
| | | GTCCTACAGTCCTCAGGACTCTACTCCCTCAGCAGCGTGG |
| | | TGACCGTGCCCTCCAGCAGCTTGGGCACCCAGACCTACAT |
| | | CTGCAACGTGAATCACAAGCCCAGCAACACCAAGGTGGAC |
| | | AAGAGAGTTGAGCCCAAATCTTGTGACAAAACTCACACAT |
| | | GCCCACCGTGCCCAGCACCTGAACTCCTGGGGGGACCGTC |
| | | AGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCGG |
| | | GAGGAGCAGTACAACAGCACGTACCGTGTGGTCAGCGTCC |
| | | TCACCGTCCTGCACCAGGACTGGCTGAATGGCAAGGAGTA |
| | | CAAGTGCAAGGTCTCCAACAAAGCCCTCCCAGCCCCCATC |
| | | GAGAAAACCATCTCCAAAGCCAAAGGGCAGCCCCGAGAAC |
| | | CACAGGTGTACACCCTGCCCCCATCCCGGGATGAGCTGAC |
| | | CAAGAACCAGGTCAGCCTGACCTGCCTGGTCAAAGGCTTC |
| | | TATCCCAGCGACATCGCCGTGGAGTGGGAGAGCAATGGGC |
| | | AGCCGGAGAACAACTACAAGACCACGCCTCCCGTGCTGGA |
| | | CTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAAGCTAGCTGA |
| 15 | nt | ATGGATCCTAAGGGCTCTCTGTCTTGGAGAATCCTGCTGT |
| | | TCCTGTCTCTGGCTTTTGAGCTGTCTTACGGCGATATCCA |
| | | GATGACACAGTCTCCTTCTTCTCTGTCTGCCTCTGTGGGC |
| | | GATAGAGTGACCATCACATGTAGAGCCTCTCAGGATATCT |
| | | CTAACTACCTGAACTGGTACCAGCAGAAGCCTGGCAAGGC |
| | | CCCAAAGCTGCTGATCTATTACACATCTAAGCTGCACTCT |
| | | GGCGTGCCTTCTAGATTTTCTGGCTCTGGATCTGGCACAG |
| | | ATTATACACTGACCATCTCTTCTCTGCAGCCTGAAGATTT |
| | | TGCTACATACTACTGTCAGCAGGGCTCTGCTCTGCCTTGG |
| | | ACCTTTGGACAGGGCACAAAGGTGGAGATCAAGCGAACTG |
| | | TGGCTGCACCATCTGTCTTCATCTTCCCGCCATCTGATGA |
| | | GCAGTTGAAATCTGGAACTGCCTCTGTTGTGTGCCTGCTG |
| | | AATAACTTCTATCCCAGAGAGGCCAAAGTACAGTGGAAGG |
| | | TGGATAACGCCCTCCAATCGGGTAACTCCCAGGAGAGTGT |
| | | CACAGAGCAGGACAGCAAGGACAGCACCTACAGCCTCAGC |
| | | AGGACCCTGACGCTGAGCAAAGCAGACTAGGAGAAACACA |
| | | AAGTCTATGCCTGCGAAGTCACCCATCAGGGCCTGAGCTC |
| | | GCCCGTCACAAAGAGCTTCAACAGGGGAGAGTGTGCTAGC |
| | | TGA |
| 16 | nt | ATGGATCCTAAGGGCTCTCTGTCTTGGAGAATCCTGCTGT |
| | | TTCTGTCTCTGGCCTTTGAGCTGTCTTACGGCCAGGTGCA |
| | | GCTGCAGGAGTCTGGACCTGGCCTGGTGAAGCCTTCTCAG |
| | | ACCCTGTCTCTGACATGTGCCGTGTACGGAGGCTCTTTTT |
| | | CTTCTGGCTACTGGAACTGGATTAGAAAGCACCCTGGAAA |
| | | GGGCCTGGAGTACATTGGCTACATCTCTTACAACGGCATC |
| | | ACATATCATAACCCTTCTCTGAAGTCTAGAATCACAATTA |
| | | ACAGAGATACATCTAAGAACCAGTACTCTCTGCAGCTGAA |
| | | CTCTGTGACACCTGAGGATACAGCCGTGTACTACTGTGCC |
| | | AGATACAAGTACGATTATGATGGCGGCCACGCTATGGATT |
| | | ACTGGGGACAGGGAACACTGGTGACCGTGTCTTCTGCTTC |
| | | TACAAAGGGCCCATCGGTCTTCCCCCTGGCGCCCTGCTCC |
| | | AGGAGCACCTCTGGGGGCACAGCGGCCCTGGGCTGCCTGG |
| | | TCAAGGACTACTTCCCCGAACCGGTGACGGTGTCGTGGAA |
| | | CTCAGGCGCCCTGACCAGCGGCGTGCACACCTTCCCGGCT |
| | | GTCCTACAGTCCTCAGGACTCTACTCCCTCAGCAGCGTGG |
| | | TGACCGTGCCCTCCAGCAGCTTGGGCACCCAGACCTACAT |
| | | CTGCAACGTGAATCACAAGCCCAGCAACACCAAGGTGGAC |
| | | AAGAGAGTTGAGCCCAAATCTTGTGACAAAACTCACACAT |
| | | GCCCACCGTGCCCAGCACCTGAACTCCTGGGGGGACCGTC |
| | | AGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCGG |
| | | GAGGAGCAGTACAACAGCACGTACCGTGTGGTCAGCGTCC |
| | | TCACCGTCCTGCACCAGGACTGGCTGAATGGCAAGGAGTA |
| | | CAAGTGCAAGGTCTCCAACAAAGCCCTCCCAGCCCCCATC |
| | | GAGAAAACCATCTCCAAAGCCAAAGGGCAGCCCCGAGAAC |
| | | CACAGGTGTACACCCTGCCCCCATCCCGGGATGAGCTGAC |
| | | CAAGAACCAGGTCAGCCTGACCTGCCTGGTCAAAGGCTTC |
| | | TATCCCAGCGACATCGCCGTGGAGTGGGAGAGCAATGGGC |
| | | AGCCGGAGAACAACTACAAGACCACGCCTCCCGTGCTGGA |
| | | CTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAAGCTAGCTGA |
| 17 | nt | ATGGATCCTAAGGGCTCTCTGTCTTGGAGAATCCTGCTGT |
| | | TCCTGTCTCTGGCTTTTGAGCTGTCTTACGGCGATATCCA |
| | | GATGACACAGTCTCCTTCTTCTCTGTCTGCCTCTGTGGGC |
| | | GATAGAGTGACCATCACATGTAGAGCCTCTCAGGATATCT |
| | | CTAACTACCTGAACTGGTACCAGCAGAAGCCTGGCAAGGC |
| | | CCCAAAGCTGCTGATCTATTACACATCTAAGCTGCACTCT |
| | | GGCGTGCCTTCTAGATTTTCTGGCTCTGGATCTGGCACAG |
| | | ATTATACACTGACCATCTCTTCTCTGCAGCCTGAAGATTT |
| | | TGCTACATACTACTGTCAGCAGGGCTCTGCTCTGCCTTGG |
| | | ACCTTTGGACAGGGCACAAAGGTGGAGATCAAGCGAACTG |
| | | TGGCTGCACCATCTGTCTTCATCTTCCCGCCATCTGATGA |
| | | GCAGTTGAAATCTGGAACTGCCTCTGTTGTGTGCCTGCTG |
| | | AATAACTTCTATCCCAGAGAGGCCAAAGTACAGTGGAAGG |
| | | TGGATAACGCCCTCCAATCGGGTAACTCCCAGGAGAGTGT |
| | | CACAGAGCAGGACAGCAAGGACAGCACCTACAGCCTCAGC |
| | | AGCACCCTGACGCTGAGCAAAGCAGACTAGGAGAAACACA |
| | | AAGTCTATGCCTGCGAAGTCACCCATCAGGGCCTGAGCTC |
| | | GCCCGTCACAAAGAGCTTCAACAGGGGAGAGTGTGCTAGT |
| | | CAGACCCCCACCAACACCATCAGCGTGACCCCCACCAACA |
| | | ACAGCACCCCCACCAACAACAGCAACCCCAAGCCCAACCC |
| | | CGCTAGTCATTTCTCAGCTTTGCAAGTGTCCCACAGATAC |
| | | CCAAGGATCCAGTCCATCAAGGTGCAGTTCACAGAGTACA |
| | | AGAAGGAGAAAGGGTTCATCCTGACATCCCAGAAAGAGGA |
| | | CGAGATCATGAAGGTCCAGAACAACAGTGTGATTATCAAC |
| | | TGCGACGGCTTCTACCTCATTAGCCTGAAGGGGTACTTTT |
| | | CTCAGGAGGTGAATATTTCCCTGCACTACCAGAAGGATGA |
| | | GGAGCCTCTCTTTCAGTTGAAGAAGGTGCGGAGCGTGAAC |
| | | AGCCTTATGGTCGCCAGCCTGACATATAAGGACAAGGTGT |
| | | ACCTGAACGTGACCACTGATAACACCAGCCTCGATGATTT |
| | | TCACGTCAACGGGGGAGAACTCATTCTGATCCACCAGAAC |
| | | CCCGGCGAATTTTGTGTCCTGGCTAGC |
| 18 | aa | HFSALQVSHRYPRIQSIKVQFTEYKKEKGFILTSQKEDEI |
| | | MKVQNNSVIINCDGFYLISLKGYFSQEVNISLHYQKDEEP |
| | | LFQLKKVRSVNSLMVASLTYKDKVYLNVTTDNTSLDDFHV |
| | | NGGELILIHQNPGEFCVL |

* highlighted AS relates to restriction linker sites

### BIBLIOGRAPHY

Aspeslaghab, Sandrine, Sophie Poste-Vinay, Sylvie Rusakiewiez, Jean-Charles Soria, Laurence Zitvogel, and Aurelien Marabelle. 2016. “Rationale for Anti-OX40 Cancer Immunotherapy Sandrine.” European Journal of Cancer 52: 50-66. https://doi.org/http://dx.doi.org/10.1016/j.ejca.2015.08.021 0959-8049/^{a}.
Buchan, Sarah L., Anne Rogel, and Aymen Al-Shamkhani. 2018. “The Immunobiology of CD27 and OX40 and Their Potential as Targets for Cancer Immunotherapy.” Blood 131 (1): 39-48. https://doi.org/10.1182/blood-2017-07-741025.
Gonzalez, Ana M., Mariana L. Manrique, Lukasz Swiech, Thomas Horn, Ekaterina Breous, Jeremy Waight, David Savitsky, et al. 2017. “INCAGN1949, an Anti-OX40 Antibody with an Optimal Agonistic Profile and the Ability to Selectively Deplete Intratumoral Regulatory T Cells.” Proceedings of the American Association for Cancer Research Annual Meeting April 1-5; W (AACR; Cancer Res 2017; 77(13 Suppl):Abstract nr 4703). https://doi.org/10.1158/1538-7445.am2017-4703.
Ito, Tomoki, Yui Hsi Wang, Omar Duramad, Shino Hanabuchi, Olivia A. Perng, Michel Gilliet, F. Xiao Feng Qin, and Yong Jun Liu. 2006. “0X40 Ligand Shuts down IL-10-Producing Regulatory T Cells.” Proceedings of the National Academy of Sciences of the United States of America 103 (35): 13138-43. https://doi.org/10.1073/pnas.0603107103.
Linch, Stefanie N., Michael J. McNamara, and William L. Redmond. 2015. “OX40 Agonists and Combination Immunotherapy: Putting the Pedal to the Metal.” Frontiers in Oncology 5 (FEB): 1-14. https://doi.org/10.3389/fonc.2015.00034.
Voo, Kui S., Laura Bover, Megan L. Harline, Long T. Vien, Valeria Facchinetti, Kazuhiko Arima, Larry W. Kwak, and Yong J. Liu. 2013. “Antibodies Targeting Human OX40 Expand Effector T Cells and Block Inducible and Natural Regulatory T Cell Function.” The Journal of Immunology 191 (7): 3641-50. https://doi.org/10.4049/jimmunol.1202752.
Zhang, Di, Anthony A. Armstrong, Susan H. Tam, Stephen G. McCarthy, Jinquan Luo, Gary L. Gilliland, and Mark L. Chiu. 2017. “Functional Optimization of Agonistic Antibodies to OX40 Receptor with Novel Fc Mutations to Promote Antibody Multimerization.” MAbs 9 (7): 1129-42. https://doi.org/10.1080/19420862.2017.1358838.

1. An OX40 activating protein comprising at least the following protein domains:
(i) an OX40 agonist antibody or an antigen-binding fragment thereof (αOX40); and,
(ii) the OX40 binding domain of OX40L (OX40L).

2. The OX40 activating protein of claim 1, wherein said OX40 agonist antibody or its antigen-binding fragment binds specifically to human OX40 and has at least one or more of the following properties:
(i) it induces the proliferation of T cells, as measured in vitro by flow cytometric analysis; or,
(ii) it induces the secretion of cytokines from T cells as measured in vitro with a CD4+ T cell activation assay.

3. The OX40 activating protein of claim 1, wherein said OX40 binding domain of OX40L is a fragment of OX40L comprising SEQ ID NO:2.

4. The OX40 activating protein of claim 1, wherein said OX40 binding domain of OX40L is fused to the C-terminus of a light or heavy chain of said OX40 agonist antibody or the antigen-binding fragment thereof.

5. The OX40 activating protein of claim 1, wherein the OX40 agonist antibody comprises a heavy and/or a light chain of an OX40 agonist IgG antibody or an antigen-binding fragment thereof.

6. The OX40 activating protein of claim 5, further comprising a peptide linker between the OX40L and the heavy and/or the light chain of said OX40 agonist IgG antibody or the antigen-binding fragment thereof.

7. The OX40 activating protein of claim 1, wherein said OX40 agonist antibody is selected from the following antibodies:
a. an antibody comprising the HCDR1 of SEQ ID NO:3, HCDR2 of SEQ ID NO:4, HCDR3 of SEQ ID NO:5, LCDR1 of SEQ ID NO:6, LCDR2 of SEQ ID NO:7 and LCDR3 of SEQ ID NO:8;
b. an antibody comprising VH and VL domains of SEQ ID NO:9 and SEQ ID NO:10 respectively;
c. an antibody that competes for binding to OX40 expressing cells with at least one of the antibodies identified in (a) or (b); or,
d. an antibody that binds to the same epitope as one of the antibodies identified in (a) or (b).

8. The OX40 activating protein of claim 6, wherein one or more antigens are fused to the heavy and/or light chain of said OX40 agonist antibody or the antigen-binding fragment thereof.

9. The OX40 activating protein of claim 8, wherein the one or more antigens include one or more viral or cancer antigens.

10. The OX40 activating protein of claim 1, comprising a light chain of the formula αOX40Light-PL1-OX40L and a heavy chain of the formula αOX40Heavy-(PL2-Ag)x, wherein
αOX40Light is a light chain of said OX40 agonist antibody;
αOX40Heavy is a heavy chain of said OX40 agonist antibody;
PL1 and PL2 are a bond or a peptide linker, and are identical or different,
Ag is one or more viral and/or cancer antigens, which are either identical or different;
x is 0, or is an integer from 1 to 20;
OX40L is the binding domain of the ligand of OX40 comprising SEQ ID NO:2 and is absent when x is 0 or is a bond.

11. A pharmaceutical composition, comprising the OX40 activating protein of claim 1 and one or more pharmaceutically acceptable excipients.

12. (canceled)

13. (canceled)

14. (canceled)

15. The OX40 activating protein of claim 2, wherein said flow cytometric analysis is an analysis of replicative dilution of CFSE-labelled cells.

16. The OX40 activating protein of claim 2, wherein said T cells are IL5, IL13, IFNγ and/or TNFα cytokines.

17. The OX40 activating protein of claim 6, wherein the peptide linker is the flexible linker FlexV1 of SEQ ID NO:13.

18. The OX40 activating protein of claim 10 wherein said PL1 and/or said PL2 is/are FlexV1 of SEQ ID NO:13.

19. The OX40 activating protein of claim 10 wherein x is 1, 2, 3, 4, or 5.

20. A method for treating or preventing cancer in a subject in need thereof comprising administering to the subject a therapeutically effective amount of the OX40 activating protein of claim 1.

21. A method for eliciting cell proliferation and/or inducing cytokine proliferation of T cells in a subject in need thereof comprising administering to the subject a therapeutically effective amount of the OX40 activating protein of claim 1.

22. A method for boosting immunity against cancer cells in a subject in need thereof suffering from cancer comprising administering to the subject a therapeutically effective amount of the OX40 activating protein of claim 1.


SEQUENCE LISTING (18 sequences)

SEQ ID NO: 1 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - protein, 142 aa, Homo sapiens
ASHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLAS

SEQ ID NO: 3 - protein, 10 aa, Artificial Sequence
  Synthetic antibody
GGSFSSGYWN

SEQ ID NO: 4 - protein, 16 aa, Artificial Sequence
  Synthetic antibody
YISYNGITYHNPSLKS

SEQ ID NO: 5 - protein, 13 aa, Artificial Sequence
  Synthetic antibody
YKYDYDGGHAMDY

SEQ ID NO: 6 - protein, 11 aa, Artificial Sequence
  Synthetic antibody
RASQDISNYLN

SEQ ID NO: 7 - protein, 7 aa, Artificial Sequence
  Synthetic antibody
YTSKLHS

SEQ ID NO: 8 - protein, 9 aa, Artificial Sequence
  Synthetic antibody
QQGSALPWT

SEQ ID NO: 9 - protein, 453 aa, Artificial Sequence
  Synthetic antibody
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAS

SEQ ID NO: 10 - protein, 216 aa, Artificial Sequence
  Synthetic antibody
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAS

SEQ ID NO: 11 - protein, 453 aa, Artificial Sequence; identical to SEQ ID NO: 9
  Synthetic antibody

SEQ ID NO: 12 - protein, 385 aa, Artificial Sequence
  Synthetic antibody
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASQTPTNTISVTPTNNSTPTNNSNPKPNPASHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLAS

SEQ ID NO: 13 - protein, 31 aa, Artificial Sequence
  Synthetic linker
ASQTPTNTISVTPTNNSTPTNNSNPKPNPAS

SEQ ID NO: 14 - DNA, 1434 bp, Artificial Sequence
  Synthetic antibody
atggatcctaagggctctctgtcttggagaatcctgctgtttctgtctctggcctttgagctgtcttacggccaggtgcagctgcaggagtctggacctggcctggtgaagccttctcagaccctgtctctgacatgtgccgtgtacggaggctctttttcttctggctactggaactggattagaaagcaccctggaaagggcctggagtacattggctacatctcttacaacggcatcacatatcataacccttctctgaagtctagaatcacaattaacagagatacatctaagaaccagtactctctgcagctgaactctgtgacacctgaggatacagccgtgtactactgtgccagatacaagtacgattatgatggcggccacgctatggattactggggacagggaacactggtgaccgtgtcttctgcttctacaaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctagctga

SEQ ID NO: 15 - DNA, 723 bp, Artificial Sequence
  Synthetic antibody
atggatcctaagggctctctgtcttggagaatcctgctgttcctgtctctggcttttgagctgtcttacggcgatatccagatgacacagtctccttcttctctgtctgcctctgtgggcgatagagtgaccatcacatgtagagcctctcaggatatctctaactacctgaactggtaccagcagaagcctggcaaggccccaaagctgctgatctattacacatctaagctgcactctggcgtgccttctagattttctggctctggatctggcacagattatacactgaccatctcttctctgcagcctgaagattttgctacatactactgtcagcagggctctgctctgccttggacctttggacagggcacaaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgtgctagctga

SEQ ID NO: 16 - DNA, 1434 bp, Artificial Sequence; identical to SEQ ID NO: 14
  Synthetic antibody

SEQ ID NO: 17 - DNA, 1227 bp, Artificial Sequence
  Synthetic antibody
atggatcctaagggctctctgtcttggagaatcctgctgttcctgtctctggcttttgagctgtcttacggcgatatccagatgacacagtctccttcttctctgtctgcctctgtgggcgatagagtgaccatcacatgtagagcctctcaggatatctctaactacctgaactggtaccagcagaagcctggcaaggccccaaagctgctgatctattacacatctaagctgcactctggcgtgccttctagattttctggctctggatctggcacagattatacactgaccatctcttctctgcagcctgaagattttgctacatactactgtcagcagggctctgctctgccttggacctttggacagggcacaaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgtgctagtcagacccccaccaacaccatcagcgtgacccccaccaacaacagcacccccaccaacaacagcaaccccaagcccaaccccgctagtcatttctcagctttgcaagtgtcccacagatacccaaggatccagtccatcaaggtgcagttcacagagtacaagaaggagaaagggttcatcctgacatcccagaaagaggacgagatcatgaaggtccagaacaacagtgtgattatcaactgcgacggcttctacctcattagcctgaaggggtacttttctcaggaggtgaatatttccctgcactaccagaaggatgaggagcctctctttcagttgaagaaggtgcggagcgtgaacagccttatggtcgccagcctgacatataaggacaaggtgtacctgaacgtgaccactgataacaccagcctcgatgattttcacgtcaacgggggagaactcattctgatccaccagaaccccggcgaattttgtgtcctggctagc

SEQ ID NO: 18 - protein, 138 aa, Homo sapiens
HFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL